(19) United States
(12) Patent Application Publication
(10) Pub. No.: US 2025/0243522 A1
(43) Pub. Date: Jul. 31, 2025

(54) ENZYMATIC PROCESS FOR THE PREPARATION OF LACTULOSE

(71) Applicant: FrieslandCampina Nederland B.V., Amersfoort (NL)

(72) Inventors: Linqiu Cao, Wagenningen (NL); Mireille Maria Gadellaa, Bemmel (NL)

(21) Appl. No.: 18/845,860

(22) PCT Filed: Mar. 10, 2023
(86) PCT No.: PCT/EP2023/056108

§ 371 Date: Sep. 10, 2024

(30) Foreign Application Priority Data
Mar. 11, 2022 (EP) 22161688.1

(51) Int. Cl. C12P 19/12; C12N 9/38; C12N 9/90

(52) U.S. Cl. CPC C12P 19/12; C12N 9/2471; C12N 9/90; C12Y 302/01023; C12Y 501/03011

(57) ABSTRACT

The invention relates to an enzymatic process for the preparation and purification of lactulose starting from lactose using cellobiose-2-epimerase (EC 5.1.3.11) followed by converting lactose to monosaccharides using lactase (EC 3.2.1.108) or to galacto-oligosaccharides using a beta-galactosidase (EC 3.2.1.23) and removing the monosaccharides, thereby resulting in a lactulose product containing, based on carbohydrate weight, at least 90 wt % lactulose, less than 1 wt % lactose, and a weight ratio of lactulose/epilactose^{>}_10.

The present invention relates to an enzymatic process for the preparation of lactulose from lactose.

Lactulose (4-O-β-D-galactopyranosyl-D-fructose) is a synthetic disaccharide that does not occur naturally and is used in the treatment of constipation and encephalopathy. It is on the list of Essential Medicines of the WHO.

Lactulose is also commonly used as prebiotic food ingredient.

The US Pharmacopoeia (USP) requires pharmaceutical grade lactulose to contain not more than 12 wt % lactose and not more than 8 wt % epilactose, both relative to lactulose.

The European Pharmacopoeia (Ph. Eur.) requires pharmaceutical grade lactulose to contain no more than 10 wt % lactose and not more than 10 wt % epilactose, relative to lactulose.

Lactulose is synthesized by the chemical or enzymatic conversion of lactose, as outlined by C. Guerroro and L. Wilson (in: Lactose-Derived Prebiotics; A Process Perspective, 2016, Chapter 5).

At present, all industrial lactulose production involves chemical synthesis. There are two main chemical synthesis routes: a Lobry de Bruyn-van Ekenstein reaction wherein the glucose residue of lactose is isomerized to fructose, and an Amadori rearrangement in which a lactosyl-amine complex is formed which is then hydrolysed to obtain lactulose. The first route is the most common and uses alkaline catalysts such as calcium hydroxide, potassium hydroxide, sodium hydroxide, or tertiary amines, optionally in the presence of borates or aluminates. Remaining lactose is subsequently removed by lactose crystallization or pressurized liquid extraction.

Disadvantages of the chemical synthesis routes are that they are not very specific, produce alkaline waste streams, the produced lactulose contains various sugar-and non-sugar contaminants and catalyst residues, and the obtained lactulose purity is only about 70-75%; the main contaminants being lactose, galactose, and epilactose. The presence of these contaminating sugars increases the caloric value of the product, and may be problematic for patients requiring a diet without these sugars.

Enzymatic routes towards lactulose have also been reported.

A first route involves the rearrangement of lactose in the presence of fructose, catalysed by a beta-galactosidase, a beta-glycosidase, or a glycosyl transferase.

Another route involves the isomerization of the glucose residue of lactose into a fructose residue using a glucose isomerase.

The most promising route, however, seems to be the direct isomerization of lactose to lactulose using a cellobiose-2-isomerase.

Unfortunately, enzymatic production results in lower yields than the chemical synthesis routes. This means that the lactulose-containing mixtures resulting from enzymatic production have high lactose content; significantly higher than chemically produced lactulose.

Although monosaccharides like galactose can be removed relatively easy by various techniques, removal of disaccharides like lactose and epilactose from lactulose is far more complicated.

As such, it is known to remove lactose from various dairy products and from galacto-oligosaccharides by enzymatic lactose hydrolysis. The problem with a similar treatment of lactulose syrups is, however, that the selectivity of these enzymes towards lactose instead of lactulose hydrolysis is generally limited, meaning that the lactulose yield will be negatively affected.

The object of the present invention is therefore the provision of an enzymatic process for the synthesis of lactulose from lactose, followed by selective removal of lactose and by-products, with no or only limited lactulose hydrolysis. A further object is to enzymatically obtain pharmaceutical grade lactulose with a quality at least similar to that of chemically produced lactulose.

This object is achieved by the process of the present invention, which requires the use of an initial lactose feed having a lactose concentration in the range 20-60 wt % and the use of specific enzymes for the lactose hydrolysis.

The process of the invention comprises the steps of:
a) providing an aqueous lactose feed with a lactose concentration in the range 20-60 wt %,
b) enzymatically converting lactose in said feed into lactulose using a cellobiose-2-epimerase enzyme, thereby forming a mixture comprising lactulose, epilactose, and lactose,
c) inactivating the cellobiose-2-epimerase enzyme,
d) enzymatically converting lactose in said mixture to either galacto-oligosaccharides (GOS) or monosaccharides using a beta-galactosidase enzyme, said conversion being conducted until the lactose content of the mixture is in the range 0.1-1 wt %, based on dry matter content, and the weight ratio lactulose/epilactose is at least 10, and
e) removing monosaccharides from said mixture, preferably by sequential simulated moving bed chromatography, thereby obtaining a purified lactulose product.

It is noted that Julio-Gonzalez et al., Separation and Purification Technology 224 (2019) 475-480, studied the further purification of a commercial, i.e. chemically produced, lactulose syrup using various β-galactosidase enzymes. The study showed that β-galactosidases from Bacillus circulans and Bifidumbacterium bifidum were more selective towards lactose hydrolysis than those derived from Kluyveromyces lactis and Aspergillus oryzae.

A 10% (w/v) solution of a commercial lactulose syrup was used as starting material. It contained 66.3 g/100 ml lactulose, 4.7 g/100 ml lactose, 3.8 g/100 ml epilactose, and 7.2 g/100 ml galactose. Based on dry matter content, this amounts to 81 wt % lactulose, 6 wt % lactose, 5 wt % epilactose, and 9 wt % galactose.

Even in the most successful experiment, 10% of the initial lactulose content was hydrolysed, while 40% of the initial epilactose content was still present.

With the process of the present invention—which involves enzymatic lactulose production starting with a lactose feed of at least 20 wt % and, consequently, starting the enzymatic lactose hydrolysis at a dry matter content of at least 20 wt %, it has been found possible to provide a higher selectivity towards to lactose and, especially, epilactose hydrolysis.

The first step of the process of the present invention involves the provision of an aqueous lactose feed with a lactose concentration in the range 20-60 wt %, preferably 20-58 wt %, more preferably 30-55 wt %, and most preferably 40-50 wt %.

This lactose feed can be an aqueous lactose solution or an aqueous suspension of lactose crystals.

The pH of the lactose feed is preferably in the range 7.0-8.5, more preferably 7.5.0-8.0

The lactose can be food grade, pharmaceutical grade, or refined.

Food grade lactose is conventionally produced by concentrating whey or whey permeate (a co-product of whey protein concentrate production) to form a supersaturated solution from which lactose crystalizes out. The lactose crystals are then removed and dried.

Refined or a pharmaceutical grade lactose can be obtained by re-dissolving the lactose crystals and treating the solution with virgin activated carbon, which absorbs a number of solutes (including riboflavin and a variety of proteins) and proteose peptones (polypeptides derived from β-casein), followed by further crystallization and washing steps.

The lactose is subsequently converted into lactulose using a cellobiose-2-epimerase enzyme. The epimerase enzyme must be able to convert the glucose-reducing end of lactose into a fructose unit. Examples of suitable epimerase enzymes are cellobiose-2-epimerases derived from thermophilic bacteria like Dictyoglomus turgidum and Caldicellulosiruptor sacchaolyticus.

The enzyme can be used as such, or in immobilized form. Various ways of enzyme immobilization are known in the art. They typically comprise a porous carrier onto which the enzyme is immobilized via covalent binding, via physical absorption (charge-charge or van der Waals interaction), via gel encapsulation, or a combination thereof. Examples of suitable solid carriers are activated acrylic polymers, preferably functionalized polymethacrylate matrices such as hexamethylenamino-functionalized polymethacrylate matrices (Sepabeads) or macroporous acrylic epoxy-activated resins like Eupergit C 250L.

Besides, carrier-free immobilized enzymes such as CLEC (cross-linked enzyme crystals) or CLEAs (cross-linked enzyme aggregates) might be also applied.

The enzymatic reaction can be suitably performed at a temperature in the range 40-80° C.

The amount of enzyme is preferably in the range 5-100 U/gram lactose, more preferably 5-50 U/gram lactose, and most preferably 10-20 U/gram lactose, wherein U stands for the lactulose isomerization unit, i.e. the amount of enzyme required to produce 1 μmole of lactulose per minute in a 50 g/l lactose solution at pH=8 and 80° C. The reaction time is preferably 5-40 hours, more preferably 8-30 hours, and most preferably 10-20 hours, depending on substrate concentration, enzyme dosage, and reaction temperature.

The resulting reaction mixture will contain lactulose, lactose, and epilactose and will have a dry solids content in the range 20-60 wt %, preferably 20-58 wt %, more preferably 30-55 wt %, and most preferably 40-50 wt % since the process started with a lactose concentration within this range. Before starting the hydrolysis step, the reaction mixture is not diluted to dry matter contents below 20 wt %, preferably not below 30 wt %, and most preferably not below 40 wt %.

In the next step, most of the lactose and epilactose is removed from this reaction mixture; either by hydrolysis of the lactose and epilactose, or by polymerizing these species to form galacto-oligosaccharides. At the same time, the lactulose should remain intact as much as possible.

Both objects require the addition of a beta-galactosidase enzyme (also called lactase enzyme).

This second step can be performed in the same reactor as the first step, provided that the epimerase enzyme is first deactivated. Such deactivation can be performed by, for instance, pH decrease (e.g. HCl addition) and/or temperature increase (e.g. boiling for at least about 100° C. for at least about 20 minutes).

A suitable lactase enzyme for use in the process of the present invention is that derived from Bifobacterium bifidum—such as the strains commercialised under the names Nola® Fit (Chr. Hansen) and Saphera® 2600L (Novozymes), or the strain commercialised under the name Nurica™ (IFF) —or derived from Lactobacillus bulgaricus—such as the strain commercialised under the name Bonlacta™ (IFF).

Suitable enzymes for GOS formation that leave the lactulose intact are derived from Bifobacterium bifidum—such as the strain commercialised under the name Nurica™ (IFF)—or derived from Bacillus circulans—such as the strain commercialised under the name Biolacta™ N5 (Amano Enzymes)—or derived from Papiliotrema terrestris. The resulting product comprising lactulose and GOS can suitably be used as prebiotic food additive, whereas the lactulose product from which the lactose has been hydrolysed is particularly suitable for pharmaceutical applications.

The dosage of the lactase enzyme depends on the reaction temperature and reaction time; higher reaction temperature and/or longer reaction times allow lower enzyme concentrations.

The enzyme dosage, reaction time, and temperature should be chosen such that (i) the lactose content is reduced to 0.1-1.0 wt %, based on dry matter, preferably 0.1-0.8 wt %, more preferably 0.1-0.6 wt %, and most preferably 0.1-0.5 wt %, and (ii) the lactulose/epilactose weight ratio is at least 10, preferably at least 11, more preferably at least 12, even more preferably at least 13, more preferably at least 14, and most preferably at least 15.

Suitable reaction conditions are 30-60° C., preferably 35-55° C., most preferably 40-50° C. to allow removal of lactose to the required extent. Reduction of the lactose content to the desired level will generally take about 1-24 hours, preferably 2-10 hours, most preferably 3-8 hours, depending on the process layout and enzyme dosage.

Low enzyme dosage requires longer reaction time. High reaction temperatures provide high reaction speed, but may also lead to enzyme denaturation.

The enzyme can be used in powder form (e.g. freeze dried, vacuum dried, or spray dried) or liquid form (e.g. dissolved in a phosphoric acid buffer solution, a tri-ethanol amine buffer solution, a tris-hydrochloric acid buffer solution, or a GOOD buffer solution).

In a specific embodiment, the enzyme is used in immobilized form. Various ways of enzyme immobilization are known in the art. They typically comprise a porous carrier onto which the beta-galactosidase is immobilized via covalent binding, via physical absorption (charge-charge or van der Waals interaction), via gel encapsulation, or a combination thereof. Besides, carrier-free immobilized enzymes such as CLEC (cross-linked enzyme crystals) or CLEA (crosslinked enzyme aggregates) might be also applied.

Carriers that can promote direct covalent binding of the enzyme are preferred, in view of their ease of operation and absence of leakage into the reaction mixture. An example of a solid carrier is an activated acrylic polymer, preferably a functionalized polymethacrylate matrix. For example, a hexamethylenamino-functionalized polymethacrylate matrix (Sepabeads) or a microporous acrylic epoxy-activated resin, like Eupergit C 250L, can be used.

The use of immobilized enzyme allows a repeated batch operating system involving several consecutive batches ('cycles'). It also allows for recycling of enzyme, which enables semi-continuous operation and multiple reuse of the enzyme.

At the end of the reaction, the enzyme may be deactivated by conventional methods, such as pH adjustment and/or temperature increase of the solution. For instance, the pH may be adjusted to about 4.5, and/or the temperature may be increased to about 72° C.

Monosaccharides formed as a result of the lactose and epilactose hydrolysis—such as glucose, galactose, fructose, and mannose—can be removed by conventional methods, such as nanofiltration (NF) or simulated moving bed chromatography (SMB), more preferably sequential simulated moving bed chromatography (SSMB). SSMB is the preferred method because SSMB usually enables high product recovery (98-99.5%) and high purity (97-99.5%). Moreover, SSMB requires less maintenance and lower water consumption than NF and consumes less solvent than non-sequential SMB.

The (sequential) SMB chromatography will be based on size exclusion. Suitable resins for such size exclusion chromatography are ion-exchange resins and gel-type resins.

With the process of the present invention it is possible to obtain a lactulose product comprising, based on total carbohydrate weight, at least 90 wt % lactulose and less than 1 wt % lactose, and having a lactulose/epilactose weight ratio of at least 10.

The lactose content is preferably 0.1-0.8 wt %, more preferably 0.1-0.6 wt %, and most preferably 0.1-0.5 wt %. The lactulose/epilactose weight ratio is preferably at least 11, more preferably at least 12, even more preferably at least 13, more preferably at least 14, and most preferably at least 15.

The monosaccharide content will be below 6 wt %, preferably below 5.5 wt %, even more preferably below 5 wt %, more preferably below 4.5 wt %, even more preferably below 4 wt %, more preferably below 3 wt %, and most preferably below 2 wt %, based on carbohydrate content.

### EXAMPLES

### Example 1: Enzymatic Lactulose Synthesis Using 2-Cellubiose Epimerase

2 grams of a 40 wt % lactose solution containing 100 mM sodium phosphate buffer and having a pH of 8 was placed on a heat shaker for 15 minutes to reach 80° C., 10 U 2-cellubiose epimerase/gram lactose was added to the solution to initialize the reaction. The reaction was followed for 48 hours by regularly taking samples and analysing the samples with Dionex HPLC (HEPAC). The lactose, lactulose, and epilactose contents—in g/L and as wt % on dry matter weight-are listed in Table 1.

TABLE 1
Enzymatic synthesis of lactulose

| Reaction | Lactose | Lactulose | Epilactose | Lactose | Lactulose | Epilactose |
|---|---|---|---|---|---|---|
| time (h) | (g/L) | (g/L)) | (g/L) | wt % | wt % | wt % |
| 0 | 395.1 | 36.6 | 33.9 | 84.6 | 7.8 | 7.3 |
| 1 | 164.5 | 221.3 | 75.7 | 35.2 | 47.4 | 16.2 |
| 24 | 114.6 | 278.4 | 53.7 | 24.5 | 59.6 | 11.5 |
| 48 | 116.7 | 273.2 | 54.2 | 25.0 | 58.5 | 11.6 |

### Example 2: Screening the Suitable Lactase Enzymes for Lactose Hydrolysis

Mixtures of lactose and lactulose were prepared which had a dry matter content of 20% (w/v), 100 mM sodium phosphate buffer, 10 mM KCl, and 2.5 mM MgCl₂ and having a pH of 5.5 and a temperature of 40° C.

The lactase hydrolysis reactions were performed with different enzymes, each with two different enzyme dosages: 10 and 100 U per gram dry solids. The reaction was performed at 20 gram scale in a plastic bottle, magnetically stirred.

Samples were taken at the indicated time intervals and analyzed by Dionex HPLC (HEPAC).

The results are listed in Table 2 and show that the enzymes derived from Aspergillus oryzae and Kluyveromyces lactis are not selective towards lactose hydrolysis and also hydrolyse most of the lactulose. It also shows that the Nola® Fit lactase enzyme has the best selectivity towards lactose hydrolysis.

TABLE 2

| | Enzyme | Reaction | | |
|---|---|---|---|---|
| Lactase | dose | time | Lactulos | Lactose |
| Aspergillus oryzae | 10 | 0 | 140.0 | 60.0 |
| (Tolerase ®) | | 1 | 60.7 | 31.7 |
| | | 2 | 40.7 | 30.7 |
| | | 4 | 30.7 | 23.6 |
| | 100 | 0 | 150.0 | 50.0 |
| | | 1 | 19.0 | 13.1 |
| | | 2 | 13.7 | 8.7 |
| | | 4 | 5.4 | 5.0 |
| Bifidobacterium bifidum | 10 | 0 | 150.0 | 50.0 |
| (Nola ® Fit) | | 1 | 149.7 | 58.9 |
| | | 2 | 151.1 | 56.9 |
| | | 4 | 149.5 | 51.0 |
| | 100 | 0 | 150.0 | 50.0 |
| | | 1 | 147.5 | 34.2 |
| | | 2 | 150.5 | 19.8 |
| | | 4 | 148.4 | 5.2 |
| Bifidobacterium bifidum | 10 | 0 | 150.0 | 50.0 |
| (Nurica ™) | | 1 | 137.9 | 49.5 |
| | | 2 | 138.4 | 39.2 |
| | | 4 | 125.5 | 24.4 |
| | 100 | 0 | 150.0 | 50.0 |
| | | 1 | 130.2 | 7.3 |
| | | 2 | 120.8 | 3.1 |
| | | 4 | 114.8 | 1.7 |
| Lactobacillus bulgaricus | 10 | 0 | 150.0 | 50.0 |
| (Bonlacta ™) | | 1 | 142.6 | 53.1 |
| | | 2 | 141.9 | 46.0 |
| | | 4 | 135.3 | 31.0 |
| | 100 | 0 | 150.0 | 50.0 |
| | | 1 | 134.5 | 12.2 |
| | | 2 | 121.3 | 1.9 |
| | | 4 | 107.4 | 1.6 |
| Kluyveromycis lactis | 10 | 0 | 150.0 | 50.0 |
| (Maxilact ®) | | 1 | 23.0 | 15.9 |
| | | 2 | 22.0 | 9.0 |
| | | 4 | 6.7 | 3.1 |
| | 100 | 0 | 150.0 | 50.0 |
| | | 1 | 2.8 | 1.4 |
| | | 2 | 3.2 | 4.2 |
| | | 4 | 3.4 | 5.4 |

### Example 3: DOE (Design of Experiment)

A Taguchi DOE was executed aiming to seek the optimum hydrolysis conditions using Nola® Fit with a dry matter content of the reaction mixture of 40 wt %.

A reaction mixture containing 68 wt % lactulose (‘Lactu’), 20 wt % lactose (‘Lact’), 12 wt % epilactose (‘Epi’); all based in dry matter.

The results displayed in Table 3 show that the lactulose/epilactose weight ratio increases with the reaction time and that ratios above 10 can be reached.

It also shows that the lactulose content relative to the total disaccharide content (Lactu/(Lact+Lactu+Epi)) increases with time and that ratio's above 90 wt % can be obtained. This means that, when the monosaccharides are removed with SSMB, a lactulose product with more than 90 wt % lactulose, less than 1 wt % lactose, and a lactulose/epilactose weight ratio>10 can be obtained.

TABLE 3

| T | pH | Enzyme | t | Gal | Glu | Man | Fru | Lact | Lactu | Epi | Lactu/ | Ratio |
|---|---|---|---|---|---|---|---|---|---|---|---|---|
| 45 | 6 | 25 | 0 | 0.0 | 0.0 | 0.0 | 0.0 | 20.3 | 67.9 | 11.8 | 67.9 | 5.8 |
| | | | 0.25 | 1.3 | 2.2 | 0.0 | 0.0 | 16.5 | 67.8 | 12.2 | 70.3 | 5.5 |
| | | | 0.5 | 2.5 | 3.9 | 0.2 | 0.0 | 14.1 | 67.6 | 11.7 | 72.4 | 5.8 |
| | | | 1 | 3.9 | 5.7 | 0.3 | 0.2 | 10.7 | 67.2 | 12.1 | 74.7 | 5.6 |
| | | | 2 | 7.5 | 9.6 | 0.8 | 0.8 | 3.9 | 66.6 | 10.8 | 81.9 | 6.2 |
| | | | 4 | 11.4 | 11.3 | 1.7 | 1.8 | 0.6 | 64.1 | 9.0 | 87.0 | 7.1 |
| | | | 6 | 13.7 | 11.5 | 2.6 | 2.9 | 0.2 | 61.7 | 7.3 | 89.2 | 8.4 |
| | | | 8 | 15.3 | 11.4 | 3.3 | 4.0 | 0.2 | 59.7 | 6.1 | 90.5 | 9.7 |
| 45 | 6.5 | 50 | 0 | 0.0 | 0.0 | 0.0 | 0.0 | 20.5 | 67.8 | 11.7 | 67.8 | 5.8 |
| | | | 0.25 | 2.5 | 3.6 | 0.2 | 0.0 | 14.2 | 67.4 | 12.1 | 71.9 | 5.6 |
| | | | 0.5 | 4.4 | 5.9 | 0.3 | 0.3 | 10.2 | 67.4 | 11.4 | 75.7 | 5.9 |
| | | | 1 | 6.3 | 8.1 | 0.6 | 0.6 | 6.0 | 67.2 | 11.2 | 79.6 | 6.0 |
| | | | 2 | 11.0 | 11.1 | 1.5 | 1.6 | 0.9 | 64.9 | 9.1 | 86.6 | 7.1 |
| | | | 4 | 15.4 | 11.2 | 2.9 | 3.5 | 0.2 | 60.0 | 6.8 | 89.6 | 8.8 |
| | | | 6 | 18.0 | 11.2 | 4.1 | 5.4 | 0.2 | 56.6 | 4.5 | 92.3 | 12.7 |
| | | | 8 | 19.9 | 11.0 | 4.8 | 7.0 | 0.2 | 53.6 | 3.5 | 93.5 | 15.3 |
| 45 | 7 | 75 | 0 | 0.3 | 0.0 | 0.0 | 0.0 | 20.1 | 67.5 | 12.1 | 67.7 | 5.6 |
| | | | 0.25 | 3.5 | 4.6 | 0.2 | 0.2 | 12.8 | 67.2 | 11.5 | 73.4 | 5.8 |
| | | | 0.5 | 5.6 | 7.2 | 0.5 | 0.5 | 8.3 | 66.8 | 11.1 | 77.5 | 6.0 |
| | | | 1 | 8.2 | 9.3 | 0.8 | 0.8 | 4.5 | 65.8 | 10.5 | 81.4 | 6.3 |
| | | | 2 | 12.3 | 10.7 | 1.8 | 2.0 | 0.6 | 65.0 | 7.7 | 88.7 | 8.5 |
| | | | 4 | 16.6 | 10.8 | 3.4 | 4.4 | 0.3 | 59.2 | 5.4 | 91.2 | 11.0 |
| | | | 6 | 19.1 | 10.6 | 4.5 | 6.6 | 0.4 | 55.6 | 3.2 | 93.9 | 17.6 |
| | | | 8 | 21.6 | 10.7 | 5.1 | 8.6 | 0.5 | 51.5 | 2.1 | 95.2 | 24.0 |
| 55 | 6 | 50 | 0 | 0.0 | 0.0 | 0.0 | 0.0 | 19.4 | 69.7 | 10.9 | 69.7 | 6.4 |
| | | | 0.25 | 2.1 | 4.5 | 0.2 | 0.2 | 12.0 | 70.0 | 11.1 | 75.2 | 6.3 |
| | | | 0.5 | 3.4 | 6.8 | 0.4 | 0.5 | 8.0 | 70.1 | 10.7 | 78.9 | 6.5 |
| | | | 1 | 5.6 | 9.2 | 0.8 | 1.0 | 3.4 | 69.7 | 10.2 | 83.7 | 6.8 |
| | | | 2 | 12.2 | 10.7 | 1.8 | 2.0 | 0.5 | 64.8 | 7.9 | 88.5 | 8.2 |
| | | | 4 | 13.1 | 10.7 | 3.4 | 4.7 | 0.3 | 62.4 | 5.4 | 91.6 | 11.5 |
| | | | 6 | 16.4 | 10.7 | 4.5 | 7.3 | 0.3 | 57.4 | 3.4 | 93.9 | 16.7 |
| | | | 8 | 18.8 | 10.6 | 5.2 | 9.6 | 0.3 | 53.2 | 2.3 | 95.3 | 23.2 |
| 55 | 6.5 | 75 | 0 | 0.3 | 0.0 | 0.0 | 0.0 | 19.6 | 68.9 | 11.2 | 69.1 | 6.1 |
| | | | 0.25 | 3.2 | 5.7 | 0.3 | 0.3 | 10.3 | 69.2 | 11.0 | 76.5 | 6.3 |
| | | | 0.5 | 4.9 | 8.2 | 0.7 | 0.6 | 5.4 | 69.5 | 10.6 | 81.3 | 6.6 |
| | | | 1 | 7.8 | 10.3 | 1.3 | 1.4 | 1.8 | 68.2 | 9.3 | 86.0 | 7.4 |
| | | | 2 | 11.5 | 10.9 | 2.5 | 3.1 | 0.4 | 64.4 | 7.1 | 89.6 | 9.0 |
| | | | 4 | 16.0 | 10.6 | 4.3 | 6.7 | 0.5 | 58.1 | 3.8 | 93.1 | 15.1 |
| | | | 6 | 19.7 | 10.8 | 5.3 | 9.8 | 0.3 | 52.0 | 2.2 | 95.4 | 23.8 |
| | | | 8 | 22.0 | 10.7 | 5.9 | 12.3 | 0.3 | 47.5 | 1.4 | 96.5 | 34.8 |
| 55 | 7 | 25 | 0 | 0.4 | 0.0 | 0.0 | 0.0 | 19.7 | 68.8 | 11.1 | 69.1 | 6.2 |
| | | | 0.25 | 1.5 | 2.3 | 0.0 | 0.0 | 15.9 | 68.7 | 11.6 | 71.4 | 5.9 |
| | | | 0.5 | 2.5 | 4.4 | 0.2 | 0.3 | 13.6 | 67.3 | 11.7 | 72.7 | 5.8 |
| | | | 1 | 3.9 | 6.8 | 0.0 | 0.0 | 9.4 | 68.2 | 11.7 | 76.3 | 5.8 |
| | | | 2 | 6.5 | 9.5 | 0.9 | 1.1 | 3.9 | 67.2 | 10.8 | 82.1 | 6.2 |
| | | | 4 | 9.7 | 11.1 | 1.7 | 2.2 | 1.1 | 65.7 | 8.6 | 87.1 | 7.6 |
| | | | 6 | 11.8 | 11.3 | 2.4 | 3.2 | 0.5 | 63.3 | 7.5 | 88.8 | 8.5 |
| | | | 8 | 13.4 | 11.3 | 3.1 | 4.2 | 0.4 | 61.3 | 6.4 | 90.0 | 9.6 |
| 65 | 6 | 75 | 0 | 0.2 | 0.0 | 0.0 | 0.0 | 20.1 | 67.8 | 12.0 | 67.9 | 5.7 |
| | | | 0.25 | 1.9 | 6.2 | 0.4 | 0.5 | 10.2 | 69.0 | 11.7 | 75.9 | 5.9 |
| | | | 0.5 | 3.0 | 8.7 | 0.8 | 1.1 | 5.6 | 69.8 | 11.1 | 80.7 | 6.3 |
| | | | 1 | 5.1 | 10.7 | 1.5 | 2.2 | 1.9 | 68.8 | 9.9 | 85.4 | 6.9 |
| | | | 2 | 7.6 | 11.5 | 2.8 | 4.2 | 0.3 | 66.0 | 7.5 | 89.4 | 8.9 |
| | | | 4 | 10.4 | 11.3 | 4.4 | 7.5 | 0.1 | 61.5 | 4.7 | 92.8 | 13.0 |
| | | | 6 | 12.4 | 11.3 | 5.2 | 9.6 | 0.0 | 58.3 | 3.3 | 94.6 | 17.8 |
| | | | 8 | 12.8 | 11.6 | 5.8 | 4.7 | 0.6 | 61.1 | 3.4 | 93.9 | 18.0 |
| 65 | 6.5 | 25 | 0 | 0.2 | 0.0 | 0.0 | 0.0 | 20.9 | 67.3 | 11.5 | 67.5 | 5.8 |
| | | | 0.25 | 1.2 | 3.1 | 0.0 | 0.0 | 16.2 | 67.8 | 11.7 | 70.8 | 5.8 |
| | | | 0.5 | 1.8 | 4.9 | 0.3 | 0.5 | 13.0 | 68.0 | 11.5 | 73.5 | 5.9 |
| | | | 1 | 2.4 | 7.1 | 0.5 | 0.8 | 9.7 | 68.4 | 11.1 | 76.7 | 6.2 |
| | | | 2 | 3.7 | 9.5 | 1.0 | 1.5 | 4.0 | 69.5 | 10.9 | 82.3 | 6.4 |
| | | | 4 | 4.4 | 10.6 | 1.3 | 2.0 | 2.3 | 69.3 | 10.1 | 84.8 | 6.9 |
| | | | 6 | 4.3 | 10.7 | 1.5 | 2.1 | 2.1 | 69.2 | 10.1 | 85.0 | 6.8 |
| | | | 8 | 4.8 | 10.5 | 1.5 | 1.0 | 2.3 | 69.7 | 10.1 | 84.9 | 6.9 |
| 65 | 7 | 50 | 0 | 0.5 | 0.0 | 0.0 | 0.0 | 21.0 | 66.9 | 11.5 | 67.3 | 5.8 |
| | | | 0.25 | 1.9 | 4.9 | 0.3 | 0.4 | 13.0 | 67.9 | 11.8 | 73.2 | 5.8 |
| | | | 0.5 | 3.0 | 7.4 | 0.5 | 0.9 | 8.9 | 67.8 | 11.5 | 76.9 | 5.9 |
| | | | 1 | 4.1 | 9.4 | 0.9 | 1.4 | 4.9 | 68.6 | 10.7 | 81.5 | 6.4 |
| | | | 2 | 4.8 | 10.5 | 1.2 | 2.0 | 2.7 | 68.1 | 10.5 | 83.8 | 6.5 |
| | | | 4 | 5.3 | 10.8 | 1.4 | 2.0 | 2.4 | 67.8 | 10.3 | 84.2 | 6.6 |
| | | | 6 | 5.3 | 10.8 | 1.3 | 2.1 | 2.0 | 68.2 | 10.2 | 84.8 | 6.7 |
| | | | 8 | 4.7 | 10.2 | 1.4 | 0.9 | 2.7 | 69.5 | 10.6 | 83.9 | 6.6 |

### Comparative Example 4

A 10% w/v solution of a commercially available lactulose syrup comprising 72 wt % lactulose, 3.38 wt % lactose, and 1.69 wt % epilactose, was converted with Nola® Fit under the conditions used in the most successful experiment of Julio-Gonzalez et al., Separation and Purification Technology 224 (2019) 475-480:30 U/g dry solids, pH 6, 40° C. In contrast to the results according to the invention of Table 3, the lactulose/epilactose weight ratio in this comparative example remained almost constant.

1. Process for the enzymatic preparation of lactulose from lactose, said process comprising the steps of:
a) providing an aqueous lactose feed with a lactose concentration in the range 20-60 wt %,
b) enzymatically converting lactose in said feed into lactulose using a cellobiose-2-epimerase enzyme, thereby forming a mixture comprising lactulose, epilactose, and lactose,
c) inactivating the cellobiose-2-epimerase enzyme,
d) enzymatically converting lactose in said mixture to either galacto-oligosaccharides (GOS) or monosaccharides using a beta-galactosidase enzyme, said conversion being conducted until the lactose content of the mixture is in the range 0.1-1 wt %, based on dry matter content, and the weight ratio lactulose/epilactose is at least 10, and
e) removing monosaccharides from said mixture, preferably by sequential simulated moving bed chromatography, thereby obtaining a purified lactulose product.

2. Process according to claim 1 wherein lactose in said mixture is converted to monosaccharides using a beta-galactosidase enzyme derived from Bifidobacterium bifidum or Lactobacillus bulgaris, preferably from Bifidobacterium bifidum.

3. Process according to claim 1 wherein lactose in said mixture is converted to GOS using a beta-galactosidase enzyme derived from Bifobacterium bifidum, Bacillus circulans, or Papiliotrema terrestris.

4. Process according to claim 1 wherein step a) provides an aqueous lactose feed with a lactose concentration in the range 20-58 wt %, preferably 30-55 wt %, most preferably 40-50 wt %.

5. Process according to claim 1 wherein step b) is conducted at a temperature in the range 40-80° C.

6. Process according to claim 1 wherein step b) is conducted using an enzyme concentration of 5-100 U/gram lactose, preferably 5-50 U/gram lactose, and most preferably 10-20 U/gram lactose.

7. Process according to claim 1 wherein step b) is conducted for 5-40 hours, preferably 8-30 hours, most preferably 10-20 hours.

8. Process according to claim 1 wherein step d) is conducted at a temperature in the range 30-60° C., preferably 35-55° C., most preferably 40-50° C.

9. Process according to claim 1 wherein step d) is conducted for 1-24 hours, preferably 2-10 hours, most preferably 3-8 hours.

10. Lactulose obtainable by the process of claim 1.

11. Lactulose product comprising, based on carbohydrate weight, at least 90 wt % lactulose, less than 1 wt % lactose, and a weight ratio of lactulose/epilactose≥10.

12. Lactulose product according to claim 11 wherein the lactulose product is obtained by a process comprising the steps of:
a) providing an aqueous lactose feed with a lactose concentration in the range 20-60 wt %,
b) enzymatically converting lactose in said feed into lactulose using a cellobiose-2-epimerase enzyme, thereby forming a mixture comprising lactulose, epilactose, and lactose,
c) inactivating the cellobiose-2-epimerase enzyme,
d) enzymatically converting lactose in said mixture to either galacto-oligosaccharides (GOS) or monosaccharides using a beta-galactosidase enzyme, said conversion being conducted until the lactose content of the mixture is in the range 0.1-1 wt %, based on dry matter content, and the weight ratio lactulose/epilactose is at least 10, and
e) removing monosaccharides from said mixture, preferably by sequential simulated moving bed chromatography, thereby obtaining a purified lactulose product.
